(19) United States
(12) Patent Application Publication
(10) Pub. No.: US 2021/0049793 A1
(43) Pub. Date: Feb. 18, 2021

(54) CORRECTING STANDARDIZED UPTAKE VALUES IN PRE-TREATMENT AND POST-TREATMENT POSITRON EMISSION TOMOGRAPHY STUDIES

(71) Applicant: KONINKLIJKE PHILIPS N.V., EINDHOVEN (NL)

(72) Inventor: Yang-Ming ZHU, WILMINGTON, MA (US)

(21) Appl. No.: 16/965,523

(22) PCT Filed: Jan. 17, 2019
(86) PCT No.: PCT/EP2019/051086

§ 371 Date: Jul. 28, 2020

Related U.S. Application Data
(60) Provisional application No. 62/625,492, filed on Feb. 2, 2018.

(51) Int. Cl. G06T 11/00; A61B 6/03; A61B 6/00

(52) U.S. Cl. CPC G06T 11/005; A61B 6/483; A61B 6/461; A61B 6/037

(57) ABSTRACT

A non-transitory computer-readable medium stores instructions readable and executable by a workstation including at least one electronic processor to perform an image interpretation method. The method includes: spatially registering first and second images of a target portion of a patient in a common image space (102), the first and second images being obtained from different image sessions and having pixel values in standardized uptake value (SUV) units; determining SUV pairs for corresponding pixels of the spatially registered first and second images (104); and controlling a display device to display a two-dimensional (2D) scatter plot of the determined SUV pairs wherein the 2D scatter plot has a first SUV axis for the first image and a second SUV axis for the second image (106).

### FIELD

The following relates generally to the medical imaging arts, positron emission tomography (PET) imaging arts, medical image interpretation arts, image reconstruction arts, and related arts.

### BACKGROUND

Currently, positron emission tomography (PET)/computed tomography (CT) imaging of cancer is a standard component of diagnosis and staging in oncology. It has also become increasingly important as a quantitative monitor of therapy response and an evaluation tool for new drug development.

To assess a patient's response to cancer therapy, clinicians read at least two sets of images (previous and current ones) and correlate findings. The use of standardized uptake values (SUV) is commonplace in clinical fludeoxyglucose (FDG) PET/CT oncology imaging, and SUV has a specific role in assessing patient response to therapy. The SUV can be calculated by:

$$S U V ( i , D , M , T ) = \frac{{v}_{i}}{\frac{D}{M} \cdot {( \frac{1}{2} )}^{\frac{t}{{t}_{1 / 2}}}} \qquad ( 1 )$$

where i is the index of a voxel of the PET image, vᵢ is the value of the voxel i (expressed as a radiotracer activity concentration in the tissue at voxel i, e.g. in units of MBq/mL or equivalent, computed from the raw pixel value based on radioactive source phantom calibration and pixel volume) in the image being converted to SUV values, D is the radiopharmaceutical dose, M is the body mass (or weight) of the patient, t is the wait time between administration of the radiopharmaceutical and the PET imaging data acquisition, and t_{1/2} is the half-life of the radiopharmaceutical.

Clinicians scroll through images and decide if the SUV of a lesion is improved or has become worse in the current image as compared with a corresponding previous image. It is known however that SUV suffers from variability due to biologic effects, patient preparation, and tracer administration. Measures have been taken to reduce and control the SUV variability. Professional societies have developed standards and guidelines (see, e.g. R. L. Wahl, H. Jacene, Y. Kasamon, and M. A. Lodge, From RECIST to PERCIST: evolving considerations for PET response criteria in solid tumors, Journal of Nuclear Medicine, vol. 50, no. 5, 122S-150S, May 2009.). Researchers have shared best practices (see, e.g. P. E. Kinahan and J. W. Fletcher, PET/CT standardized uptake values in clinical practice and assessing response to therapy, Seminars in Ultrasound, CT and MRI, vol. 31, no. 6, pp. 496-505, December 2010.). Scanner vendors have also released products (e.g. Q.Check). Despite all those efforts, SUV variability is still a concern in practice (see, e.g., M. A. Lodge, Repeatability of SUV in oncologic 18F-FDG PET, Journal of Nuclear Medicine, vol. 58, no. 4, pp. 523-532, April 2017).

Clinicians often use reference tissues to cope with the SUV variability. Aortic arch blood-pool activity or healthy liver are widely used as reference and the tumor-to-background ratio is compared in serial studies. This reference tissue approach assumes the stability of normal tissue uptake and the ratio explicitly corrects for variation in reference tissues. Variabilities in reference tissues however have been reported recently (see, e.g., R.R. Boktor, G. Walker, R. Stacey, S. Gledhill, and A.G. Pitman, Reference range for intra patient variability on blood-pool and liver SUV for 18F-FDG PET, Journal of Nuclear Medicine, vol. 54, no. 5, pp. 677-682, May 2013), which, for the blood-pool case, can be attributed to the wide spread of imaging time and individual's difference in clearance (see, e.g., J. A. Thie, Understanding SUV variability in reference tissue for 18F-FDG PET with a simple measurement model, Journal of Nuclear Medicine, vol. 55, no, 2, pp. 352-352, February 2014).

Interpretation of a PET imaging study is typically performed by a radiologist. In some clinical environments, the radiologist may be allotted only a few minutes or tens of minutes to review the current radiology study, compare with the previous radiology study, review the radiology report on the previous radiology study, and prepare and file a radiology report presenting the clinical findings of the current radiology study including comparisons with the previous radiology study. This work environment presents substantial challenges for maintaining both clinical quality and efficient throughout in radiology readings.

The following discloses new and improved systems and methods to overcome these problems.

### SUMMARY

In one disclosed aspect, a non-transitory computer-readable medium stores instructions readable and executable by a workstation including at least one electronic processor to perform an image interpretation method. The method includes: spatially registering first and second images of a target portion of a patient in a common image space, the first and second images being obtained from different image sessions and having pixel values in standardized uptake value (SUV) units; determining SUV pairs for corresponding pixels of the spatially registered first and second images; and controlling a display device to display a two-dimensional (2D) scatter plot of the determined SUV pairs wherein the 2D scatter plot has a first SUV axis for the first image and a second SUV axis for the second image.

In another disclosed aspect, a method for determining an SUV scaling shift between first and second images of a target portion of a patient obtained from different image sessions and having pixel values in standardized uptake value (SUV) units includes: spatially registering the first and second images in a common image space; determining SUV pairs for corresponding pixels of the spatially registered first and second images; determining an SUV scaling shift between the first image and the second image by performing a linear regression analysis on the determined SUV pairs in a two-dimensional (2D) space having a first SUV axis for the first image and a second SUV axis for the second image; and at least one of (i) displaying the SUV scaling shift on a display device or (ii) correcting for the SUV scaling shift by scaling SUV values of the first image or the second image in accordance with the SUV scaling shift.

In another disclosed aspect, a system includes a display device and at least one user input device. At least one electronic processor is programmed to: spatially register first and second images of a target portion of a patient in a common image space, the first and second images being obtained from different image sessions and having pixel values in standardized uptake value (SUV) units; determine SUV pairs for corresponding pixels of the spatially registered first and second images; determine an SUV scaling shift between the first image and the second image by performing a linear regression analysis on the determined SUV pairs in a two-dimensional (2D) space having a first SUV axis for the first image and a second SUV axis for the second image; correct for the SUV scaling shift by scaling SUV values of the first image or the second image in accordance with the SUV scaling shift; and control the display device to display (i) a two-dimensional (2D) scatter plot of the determined SUV pairs wherein the 2D scatter plot has a first SUV axis for the first image and a second SUV axis for the second image and (ii) the SUV scaling shift.

One advantage resides in providing a visualization device which presents an SUV plot comparing SUV values of current and previous PET imaging studies so as to assist clinicians during reading and analysis of SUV changes between the current and previous studies.

Another advantage resides in calculating and applying an SUV scaling difference correction, obviating the need to perform such scaling using manually identified reference tissues.

Another advantage resides in generating a calculated SUV scaling that is less susceptible to the variability in a single reference tissue, and less sensitive to registration errors.

Another advantage resides in generating corrected SUV values between two imaging sessions.

Another advantage resides in reducing or removing constraints or preferences that patient's follow-up studies are performed on the same scanner to control the variability, since the proposed method can correct systematic biases due to different instrumentations and algorithms.

Another advantage resides in providing linear regression approaches that are more robust than conventional linear regression techniques.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows image interpretation system according to one aspect;

FIG. 2 shows an exemplary flow chart operation of the system of FIG. 1;

FIGS. 3A and 3B show example plots of data generated by the system of FIG. 1;

FIGS. 4A and 4B show example histograms of data generated by the system of FIG. 1; and

FIGS. 5A and 5B show example histograms of data generated by the system of FIG. 1.

FIGS. 6, 7, and 8 show plots of results of linear regression tests as disclosed herein.

### DETAILED DESCRIPTION

In clinical PET, it is common to acquire images over multiple sessions with a principle purpose being to observe whether a condition (e.g. tumor, metastasis) is increasing or decreasing. To provide comparability across imaging sessions, it is known to use Standardized Uptake Values (SUV values) which normalize the counts for patient mass, radiopharmaceutical dosage, wait time, and perhaps other factors. In practice, such normalization is imperfect (for example, the assumed radiopharmaceutical dose may not match the actual dose administered to the patient, the activity level of the radiopharmaceutical may differ from its nominal value, the weight measurement may be in error, the wait time may differ from nominal, and/or so forth), and further reference is made to the SUV values in a reference region, commonly taken as the liver when it is in the field of view (FOV). However, even when this reference tissue normalization is performed there can be session-to-session SUV variability. Furthermore, when assessing the SUV changes between imaging sessions the usual practice is to display matching images from the two sessions and to visually compare, which can be subjective as it depends on the clinician's visual perception of displayed intensities as well as relies upon the clinician to detect each area where SUV has changed significantly.

In embodiments disclosed herein, the matching images are spatially registered and for each pixel the “before” and “after” SUV pair (SUV₁, SUV₂) is tabulated. In one approach, these values are plotted as x- and y-coordinates, leading to a 2D-SUV-SUV scatter plot. In the idealized situation in which there has been no change in SUV and no SUV mis-calibration between the imaging sessions, the 2D-SUV-SUV plot should be a straight line with slope=1. On the other hand, if there are regions for which SUV₂>SUV₁ then these should show up as visually observable aggregations in the plot. If there is some SUV mis-calibration then this should show up as a slope for the “unchanged” SUV value pairs that is different from 1.

In one embodiment, the 2D-SUV-SUV data pairs are generated as a matrix data structure and regression analysis is applied to determine the SUV shift correction. The linear regression slope m is the shift correction (m=1 if no shift). However, it is recognized herein that conventional linear regression is overly sensitive to spatial registration errors and undesirably depends on the regression direction. In view of this, alternative linear regression approaches are disclosed herein with substantially reduced sensitivity to mis-registration and which are symmetric with respect to the regression direction. It is noted that while these linear regression approaches are disclosed herein with illustrative application to SUV analyses as disclosed herein, the linear regression approaches disclosed herein are more generally applicable in any context in which linear regression is to be performed to fit a line to experimental data. The resulting slope m can be plotted on the 2D-SUV-SUV plot to demonstrate the shift, or alternatively one data set may be corrected for the shift, e.g. SUV₂←(1/m)*SUV₁. The shift correction m may also be reported in the radiology report, e.g. with quantitative results reported without/with the shift correction so that the clinician can evaluate all available information.

Other embodiments disclosed herein pertain to the user interface. In this aspect, the 2D-SUV-SUV plot is displayed. The user may select a region of the plot, e.g. by encircling an aggregation using the mouse pointer, and various analytical information may be generated for the selected data. One approach is to plot a histogram of slices with the value of each slice bin being the count of data in the selected region belonging to that slice. This produces a plot with slice peaks in the axial regions contributing to the selected data. Individual slices from the past and present PET imaging sessions may then be shown side-by-side to allow for visual inspection. Another presentation approach is to highlight those voxels belonging to the selected data in the displayed PET image. A clustering (i.e. connectivity) analysis may be performed to delineate a region containing the selected data. Three cross cutting planes (transverse, sagittal, and coronal) through the center of the clustered data may be displayed. Other analyses are also contemplated.

Although described herein for PET imaging systems, the disclosed approaches can be disclosed in other emission imaging modalities in which a radiopharmaceutical is administered to a patient, such as single photon emission computed tomography (SPECT) imaging systems, hybrid PET/CT or SPECT/CT imaging systems, and the like.

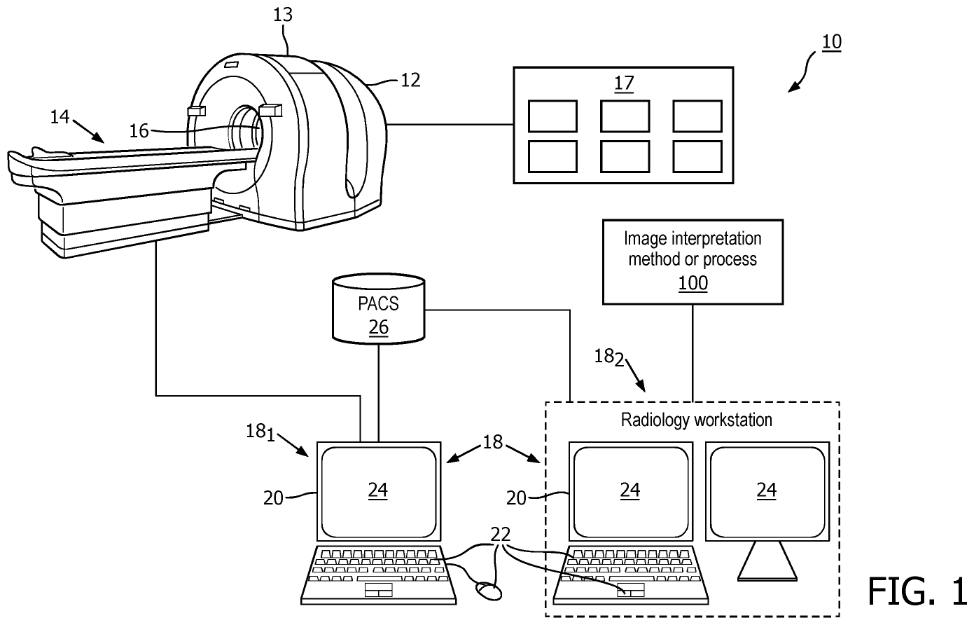

With reference to FIG. 1, an illustrative medical imaging system or device 10 is shown. As shown in FIG. 1, the system 10 includes an image acquisition device 12. In one example, the image acquisition device 12 can comprise a PET gantry of a PET/CT imaging system that further includes a computed tomography (CT) gantry 13. In other examples, the image acquisition device 12 can be a standalone PET scanner without a CT component. A patient table 14 is arranged to load a patient into an examination region 16 of the PET gantry 12 or CT gantry 13. The PET gantry 12 includes an array of radiation detectors 17.

The system 10 also includes a computer or workstation or other electronic data processing device 18 with typical components, such as at least one electronic processor 20, at least one user input device (e.g., a mouse, a keyboard, a trackball, a dictation microphone for dictating a radiology report, and/or the like) 22, and a display device 24. In some embodiments, the display device 24 can be a separate component from the computer 18. In a common clinical implementation, the at least one electronic data processing device 18 includes a first electronic data processing device 18₁ which serves as an imaging device controller (e.g. a PET scanner controller) and a second electronic data processing device 18₂ which serves as a radiology workstation. In a typical workflow, a radiology technician or other medical professional operates the PET scanner 12 using the PET controller 18₁ to acquire PET images, and the radiology images in SUV values or the information that allows to convert PET images to SUV values are stored in a Picture Archiving and Communication System (PACS) 26. The PACS may go by another nomenclature such as a Radiology Information System, RIS, or so forth.

Thereafter, a radiologist operates the radiology workstation 18₂ to perform a reading of the PET images, including retrieving (from the PACS 26) and comparing PET images from the current PET study and a previous PET study. For example, the previous PET study may have been performed before commencement of chemotherapy, radiation therapy, or other oncology therapy, while the current PET study may have been performed after such therapy. As another example, during fractionated chemotherapy or radiation therapy the previous and current PET studies may have been performed at different times during the ongoing fractionated therapy. As shown in FIG. 1, each of the PET controller 18₁ and the radiology workstation 18₂ include one or more display devices 24; the illustrative radiology workstation 18₂ includes an illustrative two displays 24, e.g. one for displaying images and the other for displaying the radiology report under draft or other textual information; display tasks may be otherwise distributed amongst the various displays 24.

The at least one electronic processor 20 is operatively connected with the one or more non-transitory storage media (not shown; such as a magnetic disk, RAID, or other magnetic storage medium; a solid state drive, flash drive, electronically erasable read-only memory (EEROM) or other electronic memory; an optical disk or other optical storage; various combinations thereof; or so forth) which stores instructions which are readable and executable by the at least one electronic processor 20 to perform disclosed operations including performing an image interpretation method or process 100. In some examples, the image interpretation method or process 100 is performed by a radiologist operating the radiology workstation 18₂, and may be performed at least in part by cloud processing.

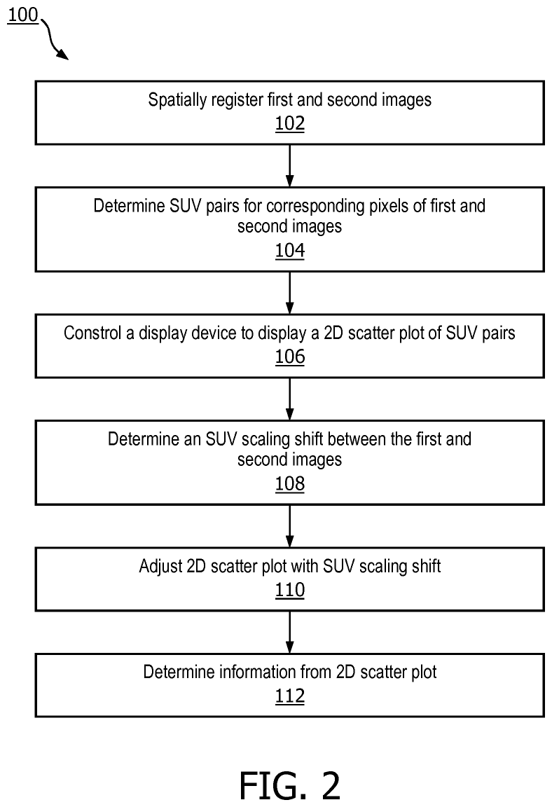

With reference to FIG. 2, an illustrative embodiment of the image interpretation method 100 is diagrammatically shown as a flowchart. Prior to commencement of the process depicted in FIG. 2, the image acquisition device 12 (e.g., the PET imaging device) is configured, or controlled by the at least one electronic processor 20 (specifically the PET controller 18₁ in the illustrative example of FIG. 1), to acquire PET imaging data, reconstruct the PET imaging data into PET images, and convert the voxel values to SUV values, e.g. using Equation (1) above which takes into account normalization information typically including the body mass or weight (M), radiopharmaceutical dose (D), and wait time (t) between administration of the radiopharmaceutical and the PET imaging data acquisition. This is done for the current PET imaging study, and was earlier done for a previous PET imaging study, and the previous and current PET imaging studies are stored in the PACS 26. At 102, the at least one electronic processor 20 (and more specifically the radiology workstation 18₂ in the illustrative example) is programmed to retrieve (from the PACS 26) and spatially register first and second images (e.g., first and second PET images) of a target portion of a patient in a common image space. As just discussed, the first and second images are typically obtained from different PET image sessions and have pixel values in SUV units. The spatial registration of the images may employ any suitable rigid or (preferably) non-rigid spatial registration technique. For example, in one approach the user manually labels corresponding landmarks in the first and second images and a spatial deformation field is applied to one image to spatially register it with the other. Additionally or alternatively the user may define contours around one or more organs, tumors, or other features of interest in the two images, and these are spatially registered. In a fully automated approach the landmarks and/or contours are identified automatically using edge and/or point detection algorithms. Images can also be automatically registered based on the image contents without the explicit feature detection. These are merely illustrative examples, and more generally any spatial registration algorithm or combination of algorithms may be employed to spatially register the first and second images.

At 104, the at least one electronic processor 20 is programmed to determine SUV pairs for corresponding pixels of the spatially registered first and second images. With the two images spatially registered, identifying corresponding pixel (or voxel) pairs is straightforward as they are spatially aligned. However, it is noted that any spatial registration algorithm is imperfect and may fail to provide perfect registry between the first and second images due to confounding factors such as changes in the size or shape of organs or tumors between the previous and current imaging sessions (e.g. tumor shrinkage or growth, bladder expansion or contraction, or so forth), rotation of organs/tumors/et cetera, or so forth.

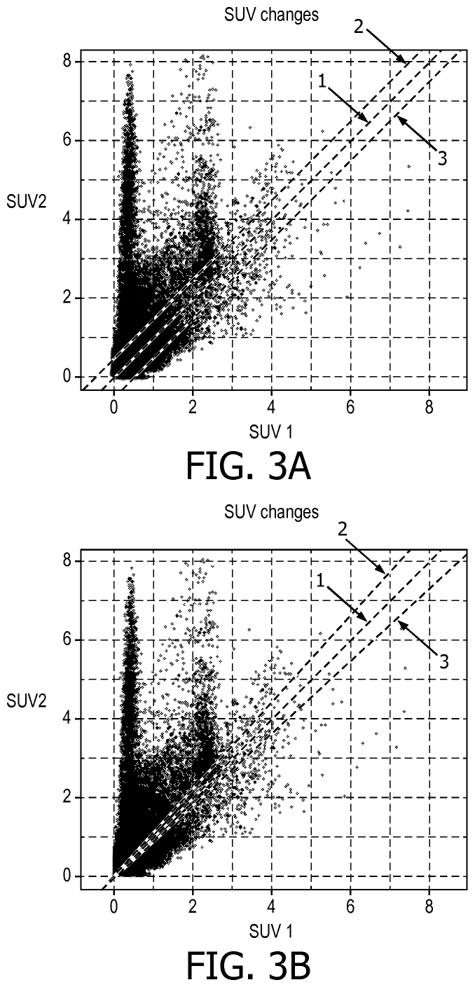

At 106, the at least one electronic processor 20 is programmed to control the display device 24 to display a two-dimensional (2D) scatter plot of the determined SUV pairs. The 2D scatter plot has a first SUV axis for the first image and a second SUV axis for the second image. FIGS. 3A and 3B show an example of such a 2D SUV-SUV plot. By this display, the radiologist can readily grasp whether the SUV values have changed significantly between the previous and current imaging studies. For example, if there are no changes then the scatter plot should line up as a line with slope m=1. On the other hand, if the SUV values have generally increased, or have generally decreased, then a substantial proportion of the points will be located above or below this line of slope m=1, with the direction (above or below) depending upon whether the SUV went up or down between the previous and current imaging studies. It should be noted that in most cases, even if the SUV has gone up or down overall, these SUV changes are typically mostly in the tumor or other malignant tissue, whereas normal tissue will likely exhibit little or no SUV change between the previous and current imaging studies. As a consequence, there will usually still be a strongly defined m=1 line corresponding to these regions of unchanged SUV, even when the SUV values of the tumor or other malignant tissue has changes substantially. Thus, the 2D-SUV-SUV plot will still typically exhibit a “reference” m=1 line that delineates the “baseline” of unchanged SUV values. Further, if the SUV scaling has changed between the previous and current imaging studies, then this “reference” line will have a slope different from m=1 with the difference being a quantification of the change in SUV scaling. All of this can be readily grasped at a glance by the radiologist viewing the 2D-SUV-SUV scatter plot.

Referring back to FIG. 2, at 108, the at least one electronic processor 20 is programmed to determine an SUV scaling shift between the first image and the second image. In some examples, the determined SUV scaling shift is displayed on the display device 24.

In some embodiments, the at least one electronic processor 20 is programmed to performing a linear regression analysis on the 2D scatter plot to determine the SUV scaling shift. In one example, the linear regression analysis adjusts a value of “m” to minimize squared distances between paired SUV coordinates to the line to be regressed, summed or averaged over the pixels “i” of the spatially registered first and second images shift. This can be performed by solving, for the SUV scaling shift (represented by “m”), Equation (1),

Σᵢxᵢyᵢm²+Σᵢ(xᵢ²−yᵢ²)m−Σᵢxᵢyᵢ=0 (1)

where xᵢ and yᵢ denote the SUV values of the SUV pair for a pixel i.

In another example, the linear regression analysis adjusts m to minimize the combined residual distances in the 2D scatter plot from each SUV pair to a line having slope m summed or averaged over the pixels i of the spatially registered first and second images. This can be performed by solving, for the SUV scaling shift (represented by “m”), Equation (2),

Σᵢxᵢ²m⁴−Σᵢxᵢyᵢm³+Σᵢxᵢyᵢm−Σᵢyᵢ²=0 (2)

where xᵢ and yᵢ denote the SUV values of the SUV pair for a pixel i. As disclosed herein, the linear regression approaches presented in Equations (1) and (2) are more robust against errors in the registration of the first and second images, as compared with traditional linear regression approaches.

At 110, the at least one electronic processor 20 is programmed to adjust or correct the 2D scatter plot with the determined SUV scaling shift to correct for the SUV scaling shift between the first image and the second image. This can be done, for example, by scaling the first SUV values (xᵢ) by the factor m to match the SUV scaling of the second SUV values (yᵢ). Alternatively, this can be done by scaling the second SUV values (yᵢ) by the factor (1/m) to match the SUV scaling of the first SUV values (xᵢ).

At 112, the at least one electronic processor 20 is programmed to determine information from the displayed 2D scatter plot. To do so, the at least one electronic processor 20 is programmed to receiving a selection of a portion of the 2D scatter plot via the user input device 22. The selection can including receiving a delineation of a region of the displayed 2D scatter plot via the user input device 22 or receiving a query defining selection criteria via the user input device. For example, the query may request selecting all pairs for which SUV₂ is at least 20% higher than SUV₁. The at least one electronic processor 20 is programmed to control the display device 24 to display a diagnostic plot of the SUV pairs of the selected portion of the 2D scatter plot. In some examples, the at least one electronic processor 20 is programmed to generate a histogram of the SUV pairs of the selected portion of the 2D scatter plot as a function of axial slice of the spatially registered first and second images. The displayed diagnostic plot comprises the histogram.

### Example

Some examples of operations 102-112 are described in more detail below. Two PET images are registered 102 to the same spatial coordinate system. The registration can be rigid or non-rigid. The PET images can be registered directly, or indirectly by registering the two associated CT images first (the PET and CT for the same study are in the same coordinate space). The registration can use the entire volume or some user-defined sub-volumes (e.g., volume of interest).

After the images are registered, the difference or ratio of the images can be computed to highlight the changes. Here, however, the changes are visualized in the 2D scatter plot or graph in operations 104 and 106. The 2D graph is easy to visualize; the difference and ratio can still be assessed on the 2D graph; and the SUV scaling difference in a serial study (that is, comparing previous and current images) can be assessed.

FIGS. 3A and 3B show SUV values from two PET images at the same spatial locations after registration. The plots shown in FIGS. 3A and 3B condense the SUVs and their relations across two PET volumes into a single 2D graph. When creating those plots, the data amount to generate the plot is optionally reduced by use of coarse images or sub-sampling the voxel grids. FIGS. 3A and 3B show the same 2D-SUV-SUV scatter plot, and differ only in terms of the superimposed lines as described herein below.

In FIGS. 3A and 3B, the line labelled “1” represents where there is no change in SUVs. The line labelled “1” has slope m=1 in this instance, but more generally may have a different slope based on the differences in SUV scaling of the previous versus current image (though this may be corrected as disclosed herein to recover slope m=1). In FIG. 3A, all the dots above the line labelled “2” indicate where SUV₂≥SUV₁+α, where α is a user configurable parameter and set to 0.5 here; all the dots below the line labelled “3” indicate where SUV₂≤SUV₁−α. The second and third lines, as well as α, serve the similar purpose of conventional difference images—they delineate area where the SUV becomes worse or better. The dots above the second line signal that at those locations the SUV becomes worse, and the dots below the third line signal that at those locations the SUV improves. (Note, this designation of “worse” and “improves” assumes a convention in which SUV₂ are the SUV values of the current PET image while SUV₁ are the SUV values of the previous PET image).

Similarly, in FIG. 3B, all the dots above the second line indicate where SUV₂≥(1+β)×SUVᵢ, where β is a user configurable parameter and set to 0.1 here. All the dots below the third line indicate where SUV₂≤(1−β)×SUV₁. The second and third lines, as well as β delineate areas where the SUV values becomes worse or better.

The user may select certain data portions depicted in the 2D-SUV-SUV scatter plot for further analysis. In one example, a user can select portion of the data from the 2D graph directly and the system performs some data analysis. As another example, user can state some numerical selection statements (e.g. “SUV₂>SUV₁+0.5 & SUV₂>2.5”), and the electronic processor 20 extracts the data that meet the criteria and performs some analysis on them.

To perform these analyses, a data selection or query is required. In one example, data selection can be done directly by picking or drawing on the 2D SUV-SUV plot. In another example, the data selection can be performed a simple selection statements (e.g., “SUV₂>SUV₁+α & SUV₂>μ” can indicate where the SUV becomes worse and “SUV₂<SUV₁−α & SUV₁>μ” can indicate where the SUV is improved, where μ is a threshold and set to 2.5, for example).

In some examples, a histogram analysis is performed in which the data points are extracted as specified by the data query and performs some analysis, e.g. histogram analysis.

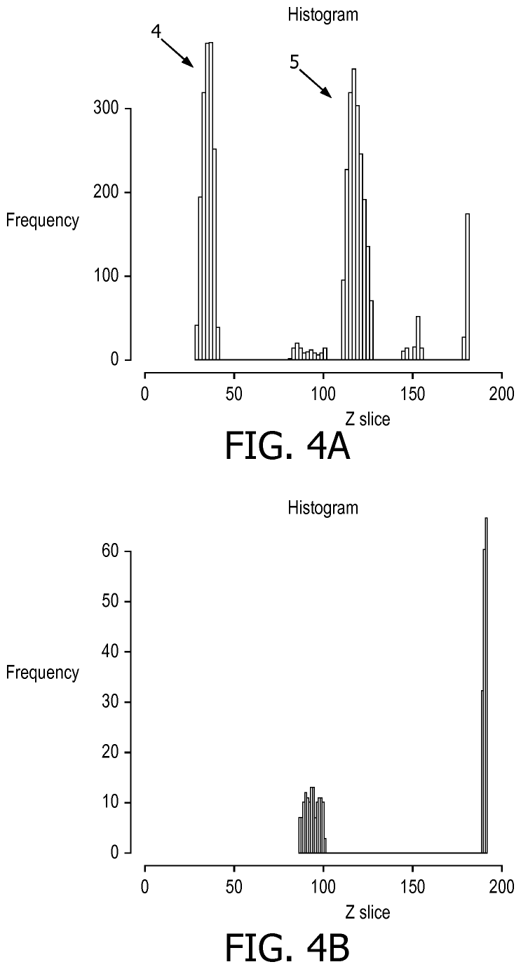

FIGS. 4A and 4B show examples of histograms. FIG. 4A shows a histogram where SUV becomes worse. These data points are aggregated by their image slice index. The peak labeled “4” corresponds to the position where the bladder is. The peak labeled “5” corresponds to the heart. Upon a user selection, (e.g. clicking on the histogram peak), the system can bring up and display those slices in both PET studies so the physicians can review and make a clinical decision.

FIG. 4B shows a histogram where SUV is improved. Again, upon user's clicking on the histogram peak, the system can bring up and display those slices in both PET studies so the physicians can review and make a clinical inference.

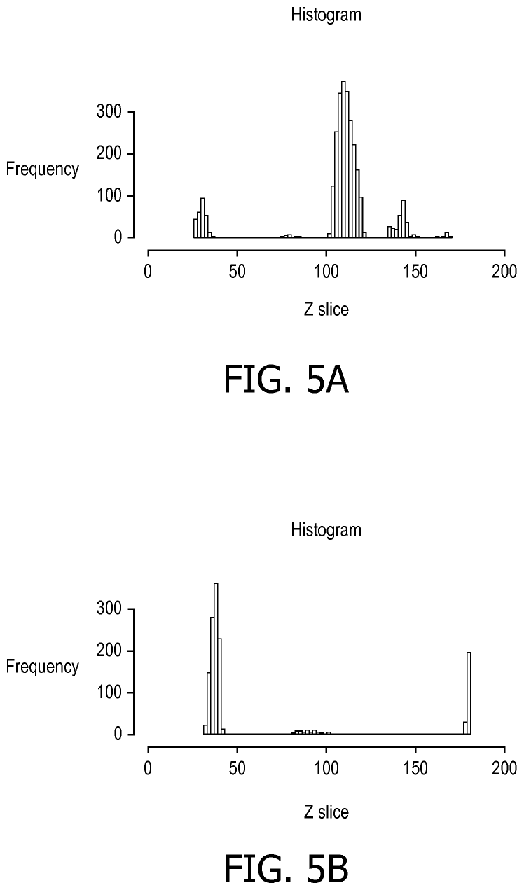

From the 2D SUV-SUV plots shown in FIGS. 3A and 3B, it is clear that in this illustrative example there are two aggregates: SUV₁ is low (around 0.5) but SUV₂ is higher; SUV₁ is around 2.5 and SUV₂ is around 3. The user can select the data in the two aggregates and the system perform some analysis. FIGS. 5A and 5B show possible results of this analysis. FIG. 5A shows from which slices the voxels form the first aggregate (associated with the heart). FIG. 5B shows from which slices the voxels form the second aggregate (associated with the bladder). Upon clicking on those peaks in histogram, the system can bring up the related slices, including, for example, multi-plane-reformatted (MPR) images, and the clinician can make a proper decision.

In some embodiments, the data points where the SUV becomes worse can be further clustered to pinpoint their locations, although the histogram analysis roughly indicates where they are. For example, voxels at which the SUV becomes worse are connected to form a bigger cluster. Small clusters, e.g. those with only one voxel, can be optionally ignored. The positions at which the SUV becomes worse form a binary volume. Segmentation tools, e.g. watershed, can be used to cluster them into different volumes of interest. The centroids of those voxels of interest are calculated. MPR planes crossing those centroids are then brought up for display, so that the clinicians can assess the SUV changes.

If the SUV₁ values and the SUV₂ values have different SUV scaling, then the slope of the data are expected to deviate from m=1. To determine the difference (if any) in SUV scaling, a regression analysis can be performed for the SUV-SUV relationship. Optionally, the regression analysis is performed after excluding the outliers where SUV is getting worse or better. For example, the data from the heart and bladder areas can be excluded from the analysis. The clinician can exclude additional regions from regression analyses based on SUV-SUV plot.

The SUV-SUV relationship is fitted using linear regression without an intercept, (i.e. SUV₂=m SUV₁), where m is a scaling correction factor. However, it is recognized herein that traditional linear regression suffers from a few difficulties in this application.

Traditional linear regression is sensitive to registration errors, and furthermore the results of traditional linear regression depend upon which SUV is chosen to be the independent variable. To remedy these issues, in more robust linear regression approaches disclosed herein the combined (or mean) squared residuals are minimized in both the x- and y-direction. Minimizing the squared distance from the paired SUV coordinates to the regression line yields Equation 1:

$${\sum }_{i} {x}_{i} {y}_{i} {m}^{2} + {\sum }_{i} ( {x}_{i}^{2} - {y}_{i}^{2} ) m - {\sum }_{i} {x}_{i} {y}_{i} = 0 \qquad ( 1 )$$

Minimizing the combined squared residuals in both x and y direction yields Equation 2:

$${\sum }_{i} {x}_{i}^{2} {m}^{4} - {\sum }_{i} {x}_{i} {y}_{i} {m}^{3} + {\sum }_{i} {x}_{i} {y}_{i} m - {\sum }_{i} {y}_{i}^{2} = 0 \qquad ( 2 )$$

To investigate the (in)sensitivity of various linear regression techniques to image spatial registration errors, two images were reconstructed from the same acquisition, but with different number of events, which are called full-dose and low-dose images. The low-dose image was reconstructed using 1/10^{th} events of the full-dose image. To study the impact of registration, one image was shifted at a step of 2 mm horizontally within the range −40 to 40 mm. The fitted traditional regression line (using SUV1 as independent variable) obtained a slope of m=0.6470 (with 0-intercept). This fitting is done under a specific condition, such as, a mis-registration error of, for example, 20 mm. By contrast, when SUV₂ is fitted to SUV₁ (i.e., use SUV₂ as an independent variable), the obtained slope was m=0.6176. In both fittings, R²=0.3996. Thus, a dependence on the choice of independent variable is seen. Moreover, the obtained slopes are much less than m=1 which would be obtained except for the imposed image shift, indicating a substantial impact of registration error on the traditional regression line.

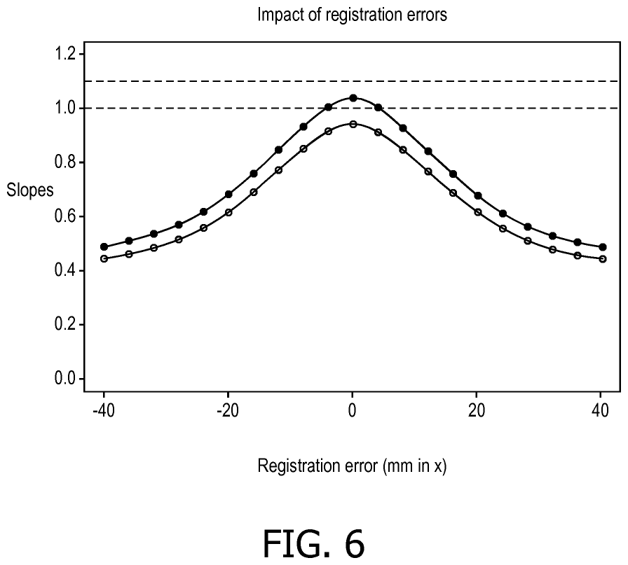

The impact of registration errors was further studied by sweeping the registration errors in the horizontal direction from −40 to 40 mm and the results are captured in FIG. 6, which supports the conclusion that the traditional linear regression is strongly impacted by the registration errors.

To remediate these issues (dependence on the choice of independent variable, and sensitivity to spatial registration errors), more robust linear regression approaches are disclosed herein (Equations (2) and (3)). The linear regression approach of Equation (2) minimizes the combined (or mean) squared residuals in both x and y directions. The linear regression approach of Equation (3) minimizes the distance from the point to the fitted line. Minimizing the square distance to the fitting line amounts to solve a quadratic equation. The objective function to minimize:

$$f ( m ) = {\sum }_{i} \frac{{( m {x}_{i} - {y}_{i} )}^{2}}{{m}^{2} + 1} \qquad ( 4 )$$

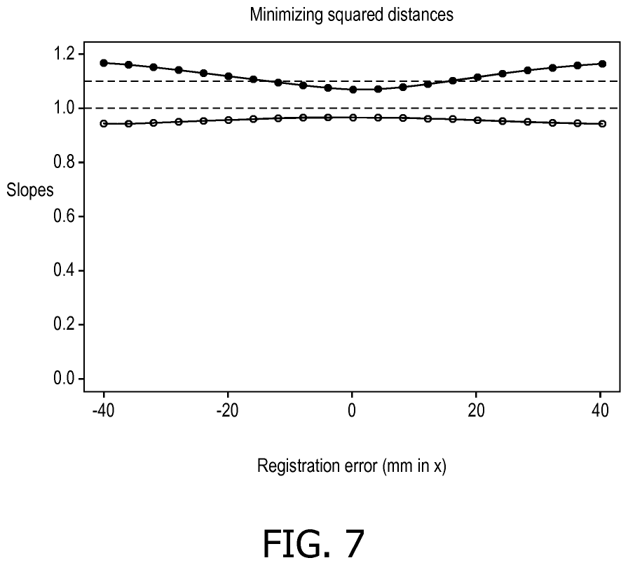

Solving Equation (4) for m leads to the quadratic Equation (2). The slopes of the fitted lines as a function of registration errors are shown in FIG. 7. Compared to the traditional linear regression (FIG. 6), the improvement against the registration errors is evident—crossing the entire range of registration errors, the slopes are in the range of 0.9498 and 0.9765, while the ground truth is 1 (open dots). When the slope is knowingly changed to 1.1, the fitted slopes are shown on the same figure (top curve with filled dots). When the roles of the SUV is switched, i.e. change which one is independent variable, the products of two slopes are invariably 1.

Minimizing the combined squared residuals in both x and y directions amounts to solve quartic equation, which has analytic solutions as well. The objective function to minimize is:

$$f ( m ) = {\sum }_{i} [ {( {y}_{i} - m {x}_{i} )}^{2} + {( {x}_{i} - \frac{1}{m} {y}_{i} )}^{2} ] \qquad ( 5 )$$

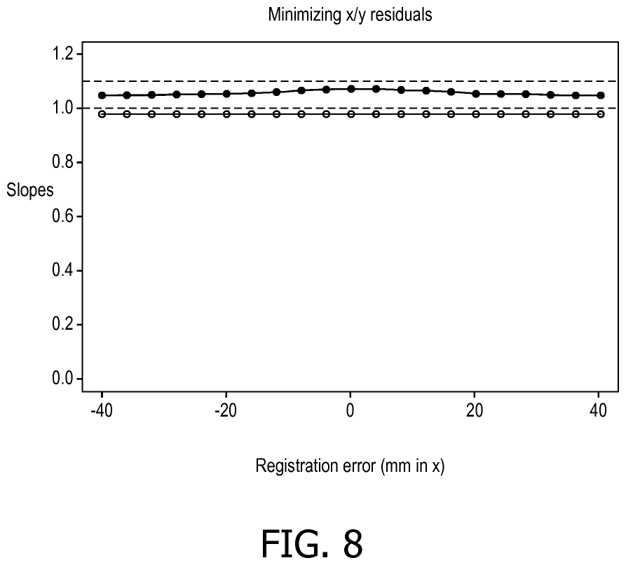

Solving Equation (5) form leads to the quartic Equation (3). The slopes of the fitted lines as a function of registration errors are shown in FIG. 8. Compared to the traditional linear regression, the improvement against the registration errors is remarkable—crossing the entire range of registration errors, the slopes are in the range of 0.9776 and 0.9851, while the ground truth is 1 (open dots). When the slope is knowingly changed to 1.1, the fitted slopes are shown on the same figure (top curve with filled dots). When the roles of the SUV is switched, i.e. change which one is independent variable, the products of two slopes are invariably 1.

It is noted that in clinical practice, protocols are followed closely with respective to variability control. Thus, in clinical practice the differences due to mis-registration is expected to be much lower than that simulated in the above examples. Moreover, as previously noted outliers along the line can be removed (i.e. “pruned”) prior to performing the linear regression. The output of the linear regression may be plotted on the 2D-SUV-SUV scatter plot as a diagonal line as an illustrated “no SUV change” line.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A non-transitory computer-readable medium storing instructions readable and executable by a workstation including at least one electronic processor to perform an image interpretation method, the method comprising:
spatially registering first and second images of a target portion of a patient in a common image space, the first and second images being obtained from different image sessions and having pixel values in standardized uptake value (SUV) units;
determining SUV pairs for corresponding pixels of the spatially registered first and second images; and
controlling a display device to display a two-dimensional (2D) scatter plot of the determined SUV pairs wherein the 2D scatter plot has a first SUV axis for the first image and a second SUV axis for the second image.

2. The non-transitory computer-readable medium of claim 1, wherein the method further includes:
determining an SUV scaling shift between the first image and the second image.

3. The non-transitory computer-readable medium of claim 2, wherein the method further includes:
displaying the SUV scaling shift.

4. The non-transitory computer-readable medium of claim 2, wherein the method further includes:
adjusting the 2D scatter plot with the determined SUV scaling shift to correct for the SUV scaling shift between the first image and the second image.

5. The non-transitory computer-readable medium of claim 4, wherein determining the SUV scaling shift includes:
performing a linear regression analysis on the 2D scatter plot to determine the SUV scaling shift.

6. The non-transitory computer-readable medium of claim 5, wherein the linear regression analysis adjusts m to minimize squared distances between SUV pairs summed or averaged over the pixels i of the spatially registered first and second images where m represents the SUV scaling shift.

7. The non-transitory computer-readable medium of claim 5, wherein the linear regression analysis is performed by solving an equation
Σᵢxᵢyᵢm²+Σᵢ(xᵢ²−yᵢ²)m−Σᵢxᵢyᵢ=0
for m, where xᵢ and yᵢ denote the SUV values of the SUV pair for a pixel i and m represents the SUV scaling shift.

8. The non-transitory computer-readable medium of claim 5, wherein the linear regression analysis adjusts m to minimize a distance in the 2D scatter plot from each SUV pair to a line having slope m summed or averaged over the pixels i of the spatially registered first and second images where m represents the SUV scaling shift.

9. The non-transitory computer-readable medium of claim 5, wherein the linear regression analysis is performed by solving an equation
Σᵢxᵢ²m⁴−Σᵢxᵢyᵢm³+Σᵢxᵢyᵢm−Σᵢyᵢ²=0
for m, where xᵢ and yᵢ denote the SUV values of the SUV pair for a pixel i and m represents the SUV scaling shift.

10. The non-transitory computer-readable medium of claim 1, wherein the method further comprises:
receiving a selection of a portion of the 2D scatter plot via a user input device (XX); and
displaying a diagnostic plot of the SUV pairs of the selected portion of the 2D scatter plot.

11. The non-transitory computer-readable medium of claim 10, wherein the receiving of the selection of the portion of the 2D scatter plot comprises one of (i) receiving a delineation of a region of the displayed 2D scatter plot and (ii) receiving a query defining selection criteria.

12. The non-transitory computer-readable medium of claim 10, further comprising:
generating a histogram of the SUV pairs of the selected portion of the 2D scatter plot as a function of axial slice of the spatially registered first and second images, wherein the displayed diagnostic plot comprises the histogram.

13. The non-transitory computer-readable medium of claim 1, wherein the first and second images are positron emission tomography (PET) images in SUV units.

14. A method for determining an SUV scaling shift between first and second images of a target portion of a patient obtained from different image sessions and having pixel values in standardized uptake value (SUV) units, the method comprising:
spatially registering the first and second images in a common image space;
determining SUV pairs for corresponding pixels of the spatially registered first and second images;
determining an SUV scaling shift between the first image and the second image by performing a linear regression analysis on the determined SUV pairs in a two-dimensional (2D) space having a first SUV axis for the first image and a second SUV axis for the second image; and
at least one of (i) displaying the SUV scaling shift on a display device or (ii) correcting for the SUV scaling shift by scaling SUV values of the first image or the second image in accordance with the SUV scaling shift.

15. The method of claim 14, wherein the linear regression analysis adjusts m to minimize squared distances between SUV pairs summed or averaged over the pixels i of the spatially registered first and second images where m represents the SUV scaling shift.

16. The method of claim 15, wherein the linear regression analysis is performed by solving an equation
Σᵢxᵢyᵢm²+Σᵢ(xᵢ²−yᵢ²)m−Σᵢxᵢyᵢ=0
for m, where xᵢ and yᵢ denote the SUV values of the SUV pair for a pixel i and m represents the SUV scaling shift.

17. The method of claim 14, wherein the linear regression analysis adjusts m to minimize a distance in the 2D scatter plot from each SUV pair to a line having slope m summed or averaged over the pixels i of the spatially registered first and second images where m represents the SUV scaling shift.

18. The method of claim 17, wherein the linear regression analysis is performed by solving an equation
Σᵢxᵢ²m⁴−Σᵢxᵢyᵢm³+Σᵢxᵢyᵢm−Σᵢyᵢ²=0
for m, where xᵢ and yᵢ denote the SUV values of the SUV pair for a pixel i and m represents the SUV scaling shift.

19. The method of claim 14, further including:
controlling a display device (XX) to display a two-dimensional (2D) scatter plot of the determined SUV pairs wherein the 2D scatter plot has a first SUV axis for the first image and a second SUV axis for the second image.

20. A system, comprising:
a display device;
at least one user input device; and
at least one electronic processor programmed to:
spatially register first and second images of a target portion of a patient in a common image space, the first and second images being obtained from different image sessions and having pixel values in standardized uptake value (SUV) units;
determine SUV pairs for corresponding pixels of the spatially registered first and second images;
determine an SUV scaling shift between the first image and the second image by performing a linear regression analysis on the determined SUV pairs in a two-dimensional (2D) space having a first SUV axis for the first image and a second SUV axis for the second image;
correct for the SUV scaling shift by scaling SUV values of the first image or the second image in accordance with the SUV scaling shift; and
control the display device to display (i) a two-dimensional (2D) scatter plot of the determined SUV pairs wherein the 2D scatter plot has a first SUV axis for the first image and a second SUV axis for the second image and (ii) the SUV scaling shift.
